(19) United States
(12) Patent Application Publication
(10) Pub. No.: US 2012/0189597 A1
(43) Pub. Date: Jul. 26, 2012

(54) NUTRITIONAL COMPOSITIONS INCLUDING EXOGENOUS NUCLEOTIDES

(75) Inventors: Norman Alan Greenberg, New Hope, MN (US); Douglas Richard Bolster, Eden Prairie, MN (US)

(73) Assignee: NESTEC S.A., Vevey (CH)

(21) Appl. No.: 13/390,175

(22) PCT Filed: Aug. 9, 2010
(86) PCT No.: PCT/US10/44852

§ 371 Date: Mar. 27, 2012

(30) Foreign Application Priority Data
Aug. 13, 2009 (US) 61233796
Aug. 28, 2009 (US) 61237709

(51) Int. Cl. A61K 31/7076; A61K 31/7068; A61K 31/7072; A61K 31/7105; A61K 38/02; A61K 36/06; A61K 35/74; A61K 38/18; A61K 38/40; C07H 21/04; A61P 1/00; C07H 19/20; C07H 19/10; A61P 21/00; A61K 36/062; A61P 37/04; A61K 31/708

(52) U.S. Cl. 424/93.42; 514/47; 514/48; 514/49; 514/51; 514/44 R; 424/195.18; 424/93.51; 424/93.5; 424/93.4; 424/93.44; 424/93.46; 514/8.9; 514/20.9; 424/94.1; 536/23.1; 536/26.7; 536/26.74; 536/26.8; 424/93.45

(57) ABSTRACT

Nutritional compositions and methods of making and using the nutritional compositions are provided. In a general embodiment, the present disclosure provides a nutritional composition including one or more exogenous nucleotides.

### BACKGROUND

The present disclosure generally relates to health and nutrition. More specifically, the present disclosure relates to nutritional compositions including exogenous nucleotides and methods of making and using the nutritional compositions.

There are many types of nutritional compositions currently on the market. Nutritional compositions can be targeted toward certain consumer types, for example, young, elderly, athletic, etc., based on the specific ingredients of the nutritional composition. Nutritional compositions can also be formulated based on the certain physiological conditions that the nutritional compositions are intended to treat or improve.

One goal of nutritional support is the renourishment of a patient so that anabolic processes for repair and growth can take place. The transition back to anabolism has relied mainly on renourishment. Due to the disease process and limitations of nutritional support, most patients that are malnourished cannot be renourished in a timely manner.

### SUMMARY

Nutritional compositions having exogenous nucleotides and methods of making and using the nutritional compositions are provided. In a general embodiment, the present disclosure provides a nutritional composition including one or more exogenous nucleotides. The nutritional composition can be a complete feeding or as an oral nutritional supplement. The nutritional composition can be in a formulation designed for any mammal such as a human or an animal. The active ingredients in the nutritional composition can also be provided as a modular product. A modular product can be defined as a method of delivering one or more specific nutrients as a supplement and not intended to be used for sole source nutrition.

The exogenous nucleotide can be in a monomeric form such as, for example, 5′ Adenosine Monophosphate (“5′-AMP”), 5′-Guanosine Monophosphate (“5′-GMP”), 5′-Cytosine Monophosphate (“5′-CMP”), 5′-Uracil Monophosphate (“5′-UMP”), 5′-Inosine Monophosphate (“5′-IMP”), 5′-Thymine Monophosphate (“5′-TMP”) or a combination thereof. The exogenous nucleotide can also be in a polymeric form such as, for example, an intact ribonucleic acid.

In an embodiment, the nutritional composition further includes one or more prebiotics. The prebiotic can be fructooligosaccharides, inulin, lactulose, galactooligosaccharides, acacia gum, soyoligosaccharides, xylooligosaccharides, isomaltooligosaccharides, gentiooligosaccharides, lactosucrose, glucooligosaccharides, pecticoligosaccharides, resistant starches, sugar alcohols or a combination thereof.

In an embodiment, the nutritional composition further includes one or more probiotics. The probiotic can be Saccharomyces, Debaromyces, Candida, Pichia, Torulopsis, Aspergillus, Rhizopus, Mucor, Penicillium, Torulopsis, Bifidobacterium, Bacteroides, Clostridium, Fusobacterium, Melissococcus, Propionibacterium, Streptococcus, Enterococcus, Lactococcus, Staphylococcus, Peptostrepococcus, Bacillus, Pediococcus, Micrococcus, Leuconostoc, Weissella, Aerococcus, Oenococcus, Lactobacillus or a combination thereof.

In another embodiment, the nutritional composition further includes one or more amino acids. The amino acid can be Isoleucine, Alanine, Leucine, Asparagine, Lysine, Aspartate, Methionine, Cysteine, Phenylalanine, Glutamate, Threonine, Glutamine, Tryptophan, Citrulline, Glycine, Valine, Proline, Serine, Tyrosine, Arginine, Histidine or a combination thereof.

In an embodiment, the nutritional composition further includes one or more synbiotics, fish oils, phytonutrients, antioxidants and/or transforming growth factor-beta.

In an embodiment, the nutritional composition is in an administerable form such as pharmaceutical formulations, nutritional formulations, dietary supplements, functional foods, beverage products or a combination thereof.

In another embodiment, the present disclosure provides a method of making a nutritional composition. The method comprises adding an exogenous nucleotide to a nutritional composition.

In an alternative embodiment, the present disclosure provides a method of minimizing protein degradation in smooth and striated muscles in a mammal. The method comprises administering to a mammal in need of same a nutritional composition including an effective amount of an exogenous nucleotide.

In yet another embodiment, the present disclosure provides a method of maintaining gastrointestinal function and structure in a mammal. The method comprises administering to a mammal in need of same a nutritional composition including an effective amount of an exogenous nucleotide.

In still another embodiment, the present disclosure provides a method of maintaining lean body mass in a mammal. The method comprises administering to a mammal in need of same a nutritional composition including an effective amount of an exogenous nucleotide.

An advantage of the present disclosure is to provide an improved nutritional composition having exogenous nucleotides.

Another advantage of the present disclosure is to provide a method of making an improved nutritional composition.

Yet another advantage of the present disclosure is to provide a nutritional composition that promotes the optimal level of protein synthesis.

Still another advantage of the present disclosure is to provide a nutritional composition that minimizes protein degradation.

Additional features and advantages are described herein, and will be apparent from the following Detailed Description.

### DETAILED DESCRIPTION

The present disclosure relates to nutritional compositions including exogenous nucleotides and methods of making and using the nutritional compositions. Embodiments of the nutritional compositions of the present disclosure can promote the optimal level of protein synthesis while minimizing protein degradation in both smooth and striated muscle for maintenance of gastrointestinal (“GI”) function and structure as well as lean body mass. The use of exogenous nucleotides may allow for a more rapid restoration of the cell energy charge and its associated benefits while continuing to restore a patient's nutritional status.

As used herein, animals include, but is not limited to mammals which includes but is not limited to rodents, aquatic mammals, domestic animals such as dogs and cats, farm animals such as sheep, pigs, cows and horses, and humans. Wherein the terms animal or mammal or their plurals are used, it is contemplated that it also applies to any animals that are capable of the effect exhibited or intended to be exhibited by the context of the passage.

As used herein, “effective amount” is preferably an amount that prevents a deficiency, treats a disease or medical condition in an individual or, more generally, reduces symptoms, manages progression of the diseases or provides a nutritional, physiological, or medical benefit to the individual. A treatment can be patient- or doctor-related. In addition, while the terms “individual” and “patient” are often used herein to refer to a human, the invention is not so limited. Accordingly, the terms “individual” and “patient” refer to any animal, mammal or human having or at risk for a medical condition that can benefit from the treatment.

As used herein, “Long term administrations” are preferably continuous administrations for more than 6 weeks.

The term “Recommended Dietary Allowance (RDA)” is preferably meant to include the nutrient level set to meet the needs of 97 to 98% of individuals, or otherwise defined as the average plus 2 standard deviations (goal intake level).

As used herein, “Short term administrations” are preferably continuous administrations for less than 6 weeks.

As used herein, a “tube feed” is preferably a complete or incomplete nutritional products that are administered to an animal's gastrointestinal system, other than through oral administration, including but not limited to a nasogastric tube, orogastric tube, gastric tube, jejunostomy tube (J-tube), percutaneous endoscopic gastrostomy (PEG), port, such as a chest wall port that provides access to the stomach, jejunum and other suitable access ports.

The cell energy charge has been proposed as an important control for the cell to favor either anabolic or catabolic processes. A patient due to either metabolic stress, nutritional stress, or both may result in a loss of nucleotides from the adenylate pool. The maintenance of the cell energy charge can attenuate the upregulation of catabolic processes resulting from metabolic stress, nutritional stress, or both which includes protein breakdown. Non limiting examples of catabolic processes that maybe effected include ubiquitin (Ub functions to regulate protein turnover in a cell by closely regulating the degradation of specific proteins by attaching to the protein and marking it for degradation), calpain (The calpain family of proteases consists of 3 well-characterized proteins, μ-calpain, m-calpain and calpastatin. Calpains are non-lysosomal calcium-dependent cysteine proteinases that selectively cleave proteins in response to calcium signals. The interaction of calcium and calpstatin regulate this signaling.) and lysosomal (organelles containing digestive enzymes that breakdown proteins).

Energy charge can be defined as: Energy charge=(ATP+V2 ADP)/(ATP+ADP+AMP) (where ATP, ADP, and AMP signify adenosine 5′-triphosphate, -diphosphate, and -monophosphate, respectively). AMP Protein Kinase (“AMPK”) is a protein that serves as a cell energy charge sensor that responds to ATP/AMP as well phosphocreatine/creatine (“PCr”/“Cr”) changing ratios for the prioritization of cellular processes based on available energy. Specifically, AMPK can target the translational control of protein synthesis as well as upregulate the ubiquitin proteosome pathway.

In a general embodiment, the present disclosure provides a nutritional composition including one or more exogenous nucleotides. The exogenous nucleotides can be in the form of monomers and polymers or a combination of monomers and polymers, as part of the nutritional compositions, for example, that serve to improve the cell energy charge and thus maintain the ability of the cell to maintain anabolic processes including protein synthesis.

A nucleotide is a subunit of deoxyribonucleic acid (“DNA”) or ribonucleic acid (“RNA”). It is an organic compound made up of a nitrogenous base, a phosphate molecule, and a sugar molecule (deoxyribose in DNA and ribose in RNA). Individual nucleotide monomers (single units) are linked together to form polymers, or long chains. Exogenous nucleotides are specifically provided by dietary supplementation.

The exogenous nucleotide can be in a monomeric form such as, for example, 5′ Adenosine Monophosphate (“5′-AMP”), 5′-Guanosine Monophosphate (“5′-GMP”), 5′-Cytosine Monophosphate (“5′-CMP”), 5′-Uracil Monophosphate (“5′-UMP”), 5′-Inosine Monophosphate (“5′-IMP”), 5′-Thymine Monophosphate (“5′-TMP”), or other monomeric nucleotides or a combination thereof. The exogenous nucleotide can also be in a polymeric form such as, for example, an intact RNA. There can be multiple sources of the polymeric form such as, for example, yeast RNA.

As used herein, the term “nutritional composition” includes, but is not limited to, complete nutritional compositions, partial or incomplete nutritional compositions, and disease or condition specific nutritional compositions. A complete nutritional composition (i.e. those which contain all the essential macro and micro nutrients) can be used as a sole source of nutrition for the patient. Patients can receive 100% of their nutritional requirements from such complete nutritional composition. A partial or incomplete nutritional composition does not contain all the essential macro and micro nutrients and cannot be used as a sole source of nutrition for the patient. Partial or incomplete nutritional compositions can be used as a nutritional supplement. A disease or condition specific nutritional composition is a composition that delivers nutrients or pharmaceuticals and can be a complete or partial nutritional composition.

The exogenous nucleotides can be combined with other ingredients for the repair and maintenance of function and structure, more specifically, nucleotides can lead to the preservation of optimal protein turnover in skeletal muscle which will result in the maintenance and/or repletion of lean body mass which in turn promotes: enhanced muscle function, mobility, activities of daily living for independence, repair, wound healing, glucose disposal, and reduction in falls. Further, the exogenous nucleotides could work more effectively to support the repair, maintenance, and improvement of skeletal muscle when used in combination with other specific food components such as amino acids (e.g., leucine), lipids to modulate inflammation (low n6:n3), bioactive peptides, protease inhibitors, creatine, etc.

As another example, the exogenous nucleotides could work more effectively to support the repair, maintenance, and improvement of smooth muscle, more specifically, the exogenous nucleotides can favor the preservation of optimal protein turnover in the smooth muscle which can result in the maintenance and/or repletion of gastrointestinal structure and function which in turn promotes: enhanced digestion, absorption, motility, barrier function, feeding tolerance and minimizes mucosal atrophy, prevent or treat mucositis, bacterial translocation, enhance immunity and infection. Further when nucleotides are used in combination with other specific food components such as amino acids (e.g., leucine), lipids to modulate inflammation (low n6:n3), bioactive peptides, protease inhibitors, creatine, etc., this effect can be maximized

In an embodiment, the nutritional composition further includes one or more prebiotics. As used herein, a prebiotic is a selectively fermented ingredient that allows specific changes, both in the composition and/or activity in the gastrointestinal microflora, that confers benefits upon host well-being and health. Non-limiting examples of prebiotics include fructooligosaccharides, inulin, lactulose, galactooligosaccharides, acacia gum, soyoligosaccharides, xylooligosaccharides, isomaltooligosaccharides, gentiooligosaccharides, lactosucrose, glucooligosaccharides, pecticoligosaccharides, resistant starches, sugar alcohols or a combination thereof.

In an embodiment, the nutritional composition further includes one or more probiotics. As used herein, Probiotics micro-organisms (hereinafter “probiotics”) are preferably microorganisms (alive, including semi-viable or weakened, and/or non-replicating), metabolites, microbial cell preparations or components of microbial cells that could confer health benefits on the host when administered in adequate amounts, more specifically that beneficially affect a host by improving its intestinal microbial balance, leading to effects on the health or well-being of the host. In general, it is believed that these micro-organisms inhibit or influence the growth and/or metabolism of pathogenic bacteria in the intestinal tract. The probiotics may also activate the immune function of the host. For this reason, there have been many different approaches to include probiotics into food products. Non-limiting examples of probiotics include Saccharomyces, Debaromyces, Candida, Pichia, Torulopsis, Aspergillus, Rhizopus, Mucor, Penicillium, Bifidobacterium, Bacteroides, Clostridium, Fusobacterium, Melissococcus, Propionibacterium, Streptococcus, Enterococcus, Lactococcus, Staphylococcus, Peptostrepococcus, Bacillus, Pediococcus, Micrococcus, Leuconostoc, Weissella, Aerococcus, Oenococcus, Lactobacillus or a combination thereof.

In another embodiment, the nutritional composition further includes one or more amino acids. Non-limiting examples of amino acids include Isoleucine, Alanine, Leucine, Asparagine, Lysine, Aspartate, Methionine, Cysteine, Phenylalanine, Glutamate, Threonine, Glutamine, Tryptophan, Citrulline, Glycine, Valine, Proline, Serine, Tyrosine, Arginine, Histidine and combinations thereof.

In an embodiment, the nutritional composition further includes one or more synbiotics, fish oils, phytonutrients and/or antioxidants. As used herein, a synbiotic is a supplement that contains both a prebiotic and a probiotic that work together to improve the microflora of the intestine. Non-limiting examples of fish oils include docosahexaenoic acid (“DHA”) and eicosapentaenoic acid (“EPA”). Non-limiting examples of phytonutrients include quercetin, curcumin and limonin. Antioxidants are molecules capable of slowing or preventing the oxidation of other molecules. Non-limiting examples of antioxidants include vitamin A, carotenoids, vitamin C, vitamin E, selenium, flavonoids, Lactowolfberry, Goji (Wolfberry), polyphenols, lycopene, lutein, lignan, coenzyme Q10 (“CoQ10”) and glutathione.

The nutritional composition can further include a transforming growth factor-beta (“TGFβ”). TGFβ is a polypeptide that can be isolated from both casein and whey milk protein fractions. TGFβ is one of a few peptides that inhibits cellular growth and proliferation to act as a cancer cell prevention agent. TGFβ also helps to maintain healthy cell survival by protecting cells from destruction by chemical insult (e.g., chemotherapy) and apoptosis. TGFβ is a potent growth inhibitor of normal epithelial cells (e.g., anti-cancer).

The nutritional composition can further include lactoferrin. Lactoferrin is a minor glycoprotein component of whey. Lactoferrin has antibacterial, antiviral, antifungal, anti-inflammatory, antioxidant and immunomodulatory activities

In another embodiment, the present disclosure provides a method of making a nutritional composition. The method comprises adding an exogenous nucleotide to a nutritional composition in an effective amount, for example, to treat a diseased condition or improve the health of the mammal. The nutritional composition can be in an administerable form such as pharmaceutical formulations, nutritional formulations, dietary supplements, functional foods, beverage products or a combination thereof.

In an alternative embodiment, the present disclosure provides a method of minimizing protein degradation in smooth and striated muscles in a mammal. The method comprises administering to a mammal in need of same a nutritional composition including an effective amount of an exogenous nucleotide. In yet another embodiment, the present disclosure provides a method of maintaining gastrointestinal function and structure in a mammal. The method comprises administering to a mammal in need of same a nutritional composition including an effective amount of an exogenous nucleotide. In still another embodiment, the present disclosure provides a method of maintaining lean body mass in a mammal. The method comprises administering to a mammal in need of same a nutritional composition including an effective amount of an exogenous nucleotide.

The nutritional composition can include the exogenous nucleotides in an amount to be administered ranging from about 0.1 mg/day to about 5.0-6.0 grams/day, including all number, whole or fractions. The exogenous nucleotides can also be administered in an amount ranging from about 0.5 gram/day to about 4.0 grams per day, from about 1 gram/day to about 3.0 grams/day, from about 1.5 grams/day to about 2.5 grams/day. The exogenous nucleotides can also be administered in an amount of about 0.5 gram/day or 1.0 gram/day or 1.5 grams/day or 2.0 grams/day or 2.5 grams/day or 3.0 grams/day or 3.5 grams/day or 4.0 grams/day or 4.5 grams/day or 5.0 grams/day or 5.5 grams/day.

By using the nutritional compositions in embodiments of the present disclosure, the preservation of optimal protein turnover in skeletal muscle can result in the maintenance and/or repletion of lean body mass which in turn promotes enhanced muscle function, mobility, activities of daily living for independence, repair, wound healing, glucose disposal, and reduction in falls. Similarly, the preservation of optimal protein turnover in smooth muscle can result in the maintenance and/or repletion of GI structure and function which in turn promotes enhanced digestion, absorption, motility, barrier function, feeding tolerance, and minimizing mucosal atrophy, bacterial translocation, and infection.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A nutritional composition comprising an exogenous nucleotide, wherein the exogenous nucleotide is selected from the group consisting of: the monomeric form of: 5′ Adenosine Monophosphate, 5′-Guanosine Monophosphate, 5′-Cytosine Monophosphate, 5′-Uracil Monophosphate, 5′-Inosine Monophosphate, 5′-Thymine Monophosphate; intact ribonucleic acid; and combinations thereof.

2. The nutritional composition of claim 1 comprising at least one component selected from the group consisting of one prebiotic; at least one prebiotic, or at least one synbiotic; and combinations thereof.

3. The nutritional composition of claim 2, wherein the prebiotic is selected from the group consisting of fructooligosaccharides, inulin, lactulose, galactooligosaccharides, acacia gum, soyoligosaccharides, xylooligosaccharides, isomaltooligosaccharides, gentiooligosaccharides, lactosucrose, glucooligosaccharides, pecticoligosaccharides, resistant starches, sugar alcohols and combinations thereof.

4. The nutritional composition of claim 2, wherein the probiotic is selected from the group consisting of Saccharomyces, Debaromyces, Candida, Pichia, Torulopsis, Aspergillus, Rhizopus, Mucor, Penicillium, Bifidobacterium, Bacteroides, Clostridium, Fusobacterium, Melissococcus, Propionibacterium, Streptococcus, Enterococcus, Lactococcus, Staphylococcus, Peptostrepococcus, Bacillus, Pediococcus, Micrococcus, Leuconostoc, Weissella, Aerococcus, Oenococcus, Lactobacillus and combinations thereof.

5. The nutritional composition of claim 1 comprising at least one amino acid.

6. The nutritional composition of claim 5, wherein the amino acid is selected from the group consisting of Isoleucine, Alanine, Leucine, Asparagine, Lysine, Aspartate, Methionine, Cysteine, Phenylalanine, Glutamate, Threonine, Glutamine, Tryptophan, Citrulline, Glycine, Valine, Proline, Serine, Tyrosine, Arginine, Histidine and combinations thereof.

7. The nutritional composition of claim 1 comprising at least one component selected from the group consisting of fish oil, at least one phytonutrient, at least one antioxidant, at least one transforming growth factor-beta; Lactoferrin; and combinations thereof.

8. The nutritional composition of claim 1, wherein the nutritional composition is a complete nutritional.

9. The nutritional composition of claim 1, wherein the nutritional composition is an incomplete nutritional.

10. The nutritional composition of claim 1, wherein the nutritional composition is a tube feed.

11. A method of modifying cell energy charge, the method comprising administering to a mammal in need of same a nutritional composition comprising an effective amount of an exogenous nucleotide.

12. The method of claim 11, wherein the exogenous nucleotide is selected from the group consisting of: the monomeric form of: 5′ Adenosine Monophosphate, 5′-Guanosine Monophosphate, 5′-Cytosine Monophosphate, 5′-Uracil Monophosphate, 5′-Inosine Monophosphate, 5′-Thymine Monophosphate; intact ribonucleic acid; and combinations thereof.

13. The method of claim 11, wherein modifying cell energy charge, results in minimizing protein degradation in smooth and striated muscles in a mammal.

14. The method of claim 11, wherein modifying cell energy charge, results in maintaining gastrointestinal function and structure in a mammal.

15. The method of claim 11, wherein modifying cell energy charge, results in maintaining lean body mass in a mammal.

16. The method of claim 11, wherein upregulation of catabolic processes is attenuated.

17. The method of claim 16, wherein the catabolic processes resulted from metabolic stress.

18. The method of claim 16, wherein the catabolic processes resulted from nutritional stress.

19. The method of claim 16, wherein the catabolic processes resulted from metabolic stress and nutritional stress.

20. The method of claim 11, wherein anabolism is favored.

21. The method of claim 11, wherein anabolism and catabolism are brought into a beneficial balance.

22. The method of claim 11, wherein protein breakdown is attenuated.

23. A method of optimizing protein synthesis in smooth and striated muscles in a mammal, the method comprising administering to a mammal in need of same a nutritional composition comprising an effective amount of an exogenous nucleotide.

24. The method of claim 23, wherein the exogenous nucleotide is selected from the group consisting of: the monomeric form of: 5′ Adenosine Monophosphate, 5′-Guanosine Monophosphate, 5′-Cytosine Monophosphate, 5′-Uracil Monophosphate, 5′-Inosine Monophosphate, 5′-Thymine Monophosphate; intact ribonucleic acid; and combinations thereof.
